(19) United States
(12) Patent Application Publication
(10) Pub. No.: US 2005/0255533 A1
(43) Pub. Date: Nov. 17, 2005

(54) Comprehensive food allergy test

(75) Inventors: Daniel C. Dantini, Ormond Beach, FL (US); Brent L. Dorval, Douglas, MA (US)

Correspondence Address: MCDERMOTT WILL & EMERY LLP, 600 13TH STREET, N.W., WASHINGTON, DC, 20005-3096, US

(73) Assignee: Brendan BioScience, LLC, Hopedale, MA (US)

(21) Appl. No.: 11/053,680

(22) Filed: Feb. 9, 2005

Related U.S. Application Data
(60) Provisional application No. 60/542,868, filed on Feb. 10, 2004.

(51) Int. Cl. G01N 33/53; G01N 33/537; G01N 33/543

(52) U.S. Cl. 435/7.92

(57) ABSTRACT

The present invention relates to methods and devices for detecting biological entities and components associated with hypersensitivity reactions in patients with food allergies. Specifically, the assays of the invention are capable of qualitatively and/or quantitatively measuring IgG, IgA, IgM and/or IgE antibodies and immunocomplexed C3b (IC-C3b) produced as a result of exposure to food.

### PRIORITY

This Application claims priority to Provisional Patent Application U.S. Ser. No. 60/542,868 filed Feb. 10, 2004; which is hereby incorporated by reference.

### BACKGROUND OF THE INVENTION

1. Field of the Invention

The present invention relates to methods and devices for detecting biological entities and components associated with hypersensitivity reactions in patients with food allergies.

2. Background

It is well established that food-related allergies cause a variety of illnesses, whether it be in humans or other animals. Approximately two percent of adults and about five percent of infants and young children in the United States suffer from food allergies and each year, roughly 30,000 individuals require emergency room treatment as a result of food allergies. At present, there is no cure for food allergies and a food allergic consumer must avoid the food to which the consumer is allergic. The timing and location of an allergic reaction to food is affected by digestion. For example, an allergic person may first experience a severe itching of the tongue or “tingling lips.” Vomiting, cramps or diarrhea may follow. Later, as allergens enter the bloodstream and travel throughout the body, they can cause a drop in blood pressure, hives or eczema, or asthma when they reach the lungs. The onset of these symptoms may vary from a few minutes to an hour or two after the food is eaten. Delayed reactions take hours or days to manifest symptoms.

Von Pirquet first described serum sickness, the prototype of Immune Complex disease in 1925. Any food protein entering the circulation in sufficient quantity can produce symptom patterns resembling serum sickness. If antigens make it into the blood stream, they can stimulate the production of antibodies. These antibodies can then combine with antigens in the blood stream to produce circulating immune complexes (CICs).

Food-enriched blood, coming from the gastrointestinal tract (GIT), goes through the liver where most immune-complexes are removed. If circulating complexes pass the liver filter, they may cause disturbances in many organs. The other path of absorption of molecules from the GIT is through lymphatic drainage. The lymph channels flow together to form the thoracic duct, a flimsy vessel which drains its contents into the subclavian vein. This pathway may direct antigenic molecules directly to the lungs where food antigens may excite intrinsic asthmatic attacks, bronchitis, or more serious and enigmatic inflammatory lung diseases. The combination of antibody with antigen in the blood stream is a circulating immune complex (CIC). In most cases, CICs are simply removed from the circulation by macrophages prior to triggering a cascade of events which may cause multiple symptoms, and possibly tissue damage.

CIC's activate complement which is a circulating system of 25 proteins which interact to produce a variety of defensive molecular weapons. There are two main functions of the complement cascade. The first is to opsonize bacteria, viruses and antibodies with covalently bound C3b. The bacterial, viral or CIC-C3b complex is bound to the CR1 receptors through the ligand C3b. CR1 receptors are found on red blood cells or other cells, such as macrophages which results in rapid removal of the C3b-CICs. The CR1 receptor is a cofactor that causes rapid degradation of C3b by Factor H and Factor I to CIC-C3bi and ultimately to CIC-C3d/C3d,g. It is noteworthy that C3d/C3d,g contain a thioester bond, which causes this fragment to remain covalently bound to the activator i.e. CIC, indefinitely. The second function is to lyse cells by activation of the terminal pathway proteins C5 through C9. C5-C9 attach to cell surfaces, assemble into pores (membrane attack complex), and disrupt the cell membrane or cell walls. The net effect is that ions and water flow into a cell causing the cell to burst.

Clearly the inadvertent or inappropriate activation of complement can have serious consequences for healthy self-cells and tissues. CIC's leave capillaries to trigger inflammatory events in target tissues. A classic model of complex-induced pathology is the Arthus reaction, which appears 3-6 hours after antigen challenge and involves large insoluble complexes with complement (C3b) passing through vessel walls to excite inflammatory responses in target tissues.

Regardless of the animal, allergens (antigens) from food, food additives or environmental sources cause an acquired immunity. Acquired immunity is simply the ability of allergens to either cause the production of antibodies (IgM, IgA, IgG, IgE and IgD) or interact with the mucosa or epidermis and stimulate T-cells. These antibodies react with the allergen and cause symptoms associated with allergy. Allergic reactions are classified into four types (I, II, III, IV) based on the Gell/Coombs scheme.

Immunoenzymometric assays involve the binding of an analyte of interest with a reaction or binding partner, where the binding partner carries a label. The binding partner is contained in a test strip, well or other apparatus so that it is non-reactive unless and until its partner analyte contacts the test strip. When this happens, the analyte and labelled binding partner bind to each other, forming a complex. This is accomplished by reacting the label carried by the binding partner with another substance, to form a detectable signal. When the label is an enzyme, as it frequently is, the substance is a substrate for the enzyme. The substrate for the enzyme either forms a visible color or changes color. Measuring the change or amount of color provides a measure of the produced complex, and hence of the analyte.

There is a need for quick, accurate, simple assays that can be performed by laboratory personnel as well as by non-technical personnel outside of a laboratory setting to test biological fluids of organisms to determine the presence of biological analytes such as allergen-specific immunoglobulins and immunocomplexes in the blood that are associated with or indicative of food, food additive or chemical allergies. Specifically, there is a need to test biological fluids of patients to determine whether they are experiencing type I, II, III or IV hypersensitivity.

All publications, scientific, patent or otherwise are hereby incorporated by reference in their entirety for all purposes.

### SUMMARY OF THE INVENTION

One aspect of the invention relates to a kit for determining the presence of allergen-specific immunoglobulins and immuncomplex C3b in a biological sample comprising: a solid support comprising an immobilized allergen that is to be exposed to a biological sample thereby binding and immobilizing allergen-specific immunoglobulins and immuncomplex C3b; and at least two labeled binding partners a first labeled binding partner that specifically binds the immobilized allergen-specific immunoglobulins; and a second labeled binding partner that specifically binds the immobilized immunocomplex C3b (IC-C3b).

In one embodiment of this aspect of the invention the biological sample is serum or saliva. In another embodiment, the solid support is a microtiter dish well. In yet a further embodiment, the first labeled binding partner is selected from the group consisting of labeled anti-human, anti-IgG, anti-IgA, and anti-IgM antibodies and said second labeled binding partner is an anti-C3d antibody. In still another embodiment, the first labeled binding partner is anti-IgG antibody and the second labeled binding partner is an anti-C3d antibody. In another embodiment, the label is part of a signal producing system. In a further embodiment, the amount of label immobilized on the solid support can be read quantitatively. In still another embodiment, the allergen is derived from the group consisting of milk, corn, shrimp, lobster, crab, peanuts, walnuts, fish, eggs, soy and wheat. In yet a further embodiment, the allergen is selected from the group consisting of monosodium glutamate (MSG), gluten, casein, β-lactoglobulin and bovine serum albumin.

Another aspect of the invention relates to a method of determining the presence of allergen-specific immunoglobulins and immuncomplex C3b in a biological sample comprising: exposing a solid support comprising an immobilized allergen to a biological sample; washing unbound molecules from the biological samples from the solid support; exposing said solid support to at least two labeled binding partners: a first labeled binding partner that specifically binds the immobilized allergen-specific immunoglobulins; and a second labeled binding partner that specifically binds the immobilized immuncomplex C3b; washing unbound labeled binding partners from the solid support; detecting the presence of label bound to the solid support; and correlating it with the presence of allergen specific IC-C3b and allergen specific immunoglobulins in the biological sample.

In one embodiment of this aspect of the invention, the biological sample is serum or saliva. In another embodiment, the solid support is a microtiter dish well. In a further embodiment, the first labeled binding partner is selected form the group consisting of labeled anti-human, anti-IgG, anti-IgA, and anti-IgM antibodies and said second labeled binding partner is an anti-C3d antibody. In yet another embodiment, the first labeled binding partner is anti-IgG antibody and said second labeled binding partner is an anti-C3d antibody. In still another embodiment, the label is part of a signal producing system. In yet a further embodiment, the amount of label immobilized on the solid support is be read quantitatively. In another embodiment, the allergen is derived from the group consisting of milk, corn, shrimp, lobster, crab, peanuts, walnuts, fish, eggs, soy and wheat. In yet a further embodiment, the allergen is selected from the group consisting of monosodium glutamate (MSG), gluten, casein, β-lactoglobulin and bovine serum albumin.

Another aspect of the invention relates to a test strip apparatus for determining the presence of allergen-specific immunoglobulins and immuncomplex C3b in a biological sample comprising a biblious substrate comprising a first zone comprising at least two diffusible labeled receptors: a first diffusible labeled receptor that specifically binds the allergen-specific IgE, IgG, IgA and/or IgM; and a second diffusible labeled receptor that specifically binds C3b; a second zone comprising at least one area wherein each area has at least one immobilized allergen; and a third zone comprising an immobilized second receptor specific for said first and/or said second diffusible labeled receptor; located in sequence in a capillary fluid flow direction in said test strip apparatus; and wherein an accumulation of label in the second zone correlates with the presence of, and is proportional to an amount of allergen specific IC-C3b and allergen specific immunoglobulins in the biological sample.

In one embodiment of this aspect of the invention, the biological sample is serum or saliva. In another embodiment, the biblious substrate a nitrocellulose membrane. In a further embodiment, the first diffusible labeled receptor is labeled anti-IgG antibody and said second diffusible labeled receptor is anti-C3d antibody. In yet another embodiment, the label comprises latex particles. In a further embodiment, the label comprises colloidal gold particles. In still another embodiment, the at least one allergen is derived from the group consisting of milk, corn, shrimp, lobster, crab, peanuts, walnuts, fish, eggs, soy and wheat. In yet another embodiment, the at least one allergen is selected from the group consisting of monosodium glutamate (MSG), gluten, casein, β-lactoglobulin and bovine serum albumin. In still another embodiment, the second zone comprises a plurality of areas that are stripes of different immobilized allergens. In a further embodiment, the first diffusible labeled receptor of labeled binding partner is gold-conjugated goat anti-human IgG antibody and said second diffusible labeled receptor is gold-conjugated goat anti-human C3d antibody. In a another embodiment, the immobilized second receptor specific for the diffusible labeled receptors is a mouse generated anti-goat antibody.

Another aspect of the invention relates to a method of determining the presence of allergen-specific immunoglobulins and immuncomplex C3b in a biological sample comprising: a first zone comprising at least two diffusible labeled receptors: a first diffusible labeled receptor that specifically binds the allergen-specific IgE, IgG, IgA and/or IgM; and a second diffusible labeled receptor that specifically binds C3b; a second zone comprising at least one area wherein each area has at least one immobilized allergen; and a third zone comprising an immobilized second receptor specific for said first and/or said second diffusible labeled receptor; located in sequence in a capillary fluid flow direction in said test strip apparatus; and allowing said biological fluid to migrate up the test strip apparatus by capillary action; and reading said test strip by correlating the presence of label accumulation in said second area with the presence of allergen specific IC-C3b and allergen specific immunoglobulins in the biological sample.

In one embodiment, the biological sample is serum or saliva. In another embodiment, said biblious substrate a nitrocellulose membrane. In a further embodiment, the at least two labeled binding partners are labeled anti-IgG antibodies and anti-immunocomplex C3d antibodies. In still another embodiment, the label comprises latex particles. In yet another embodiment, the label comprises colloidal gold particles. In still a further embodiment, the at least one allergen is derived from the group consisting of milk, corn, shrimp, lobster, crab, peanuts, walnuts, fish, eggs, soy and wheat. In another embodiment, the at least one allergen is selected from the group consisting of monosodium glutamate (MSG), gluten, casein, β-lactoglobulin and bovine serum albumin. In a further embodiment, the second zone comprises a plurality of areas that are strips of immobilized different allergens. In still a further embodiment, the first labeled binding partner is gold conjugated goat anti-human IgG antibody and said second diffusible labeled receptor is gold-conjugated goat anti-human C3d antibody. In yet another embodiment, the immobilized second receptor specific for the diffusible labeled receptors is a mouse generated anti-goat antibody.

Another aspect of the invention relates to using the method and devices described herein to diagnose hypersensitivity reactions wherein wherein a presence of allergen-specific IgE and a substantial lack of allergen-specific IgG, IgA, IgM and IC-C3b in the biological sample correlates with Type I hypersensitivity reactions; a presence of allergen-specific IgG, IgA, and IgM and IC-C3b and a substantial lack of allergen-specific IgE in the biological sample correlates with Type II hypersensitivity reactions; a presence of allergen-specific IgG and IC-C3b and a substantial lack of allergen-specific IgE, IgA, IgM in the biological sample correlates with Type I hypersensitivity reactions; and a substantial lack of allergen-specific IgG, IgA, IgM and IC-C3b in the biological sample correlates with Type I hypersensitivity reactions.

### DETAILED DESCRIPTION OF THE INVENTION

The invention relates to methods, kits and apparatuses for the detection and determination of antibodies and/or immune complexes that bind to allergens in foods, chemicals, and food additives. Little information exists on sensitivity to the ingested products and foods except for IgE related reactions. It is shown herein that, not only IgE, but also a combination of other antibodies (IgG, IgA, IgM and immune complex) can cause food allergen associated disease.

“Allergens,” as used herein, relate to substances that cause allergies. Allergens may be from food, chemicals or food additives. Structurally speaking, allergens may range in size from small and simple chemical compounds to polypeptides and other biological macromolecules. Food allergens are commonly found in e.g., Apple, Corn, Oat, Soybean, Baker's Yeast, Cottonseed, Onion, Strawberry, Banana, Cow's Milk, Orange, Sunflower Seed, Beef, English Walnut, Peanut, Tea, Beet, Garlic, Pork, Tomato, Brewer's Yeast, Grapefruit, Red Pepper, Tuna, Broccoli, Green Olive, Rice, Turkey, Cocao, Hops, Rye, White Potato, Cocoanut, Lemon, Safflower Seed, White Seedless Grape, Coffee, Mushroom, Sesame, Whole Egg (Chicken), Cola Nut, Mustard, Sole, Whole Wheat, Almond, Cherry Green Pea, Pineapple, Apricot, Chicken, Honeydew Melon, Pinto Bean, Barley, Chili Pepper, Lamb, Pumpkin, Basil, Cinnamon, Lettuce, Salmon, Beet, Clam, Lima Bean, Scallops, Cabbage, Crab, Lobster, Shrimp, Cantaloupe, Cranberry, Millet, Squash Mix, Carrot, Cucumber, Oregano, Sweet Potato, Cashew Nut, Dill Seed, Peach, Tumeric, Cauliflower, Ginger, Pear, Vanilla, Celery, Green Bean, Pecan, and Watermelon. Common food additives such as monosodium glutamate (MSG) and gluten are also known to be allergens. Children typically outgrow their allergies to milk, egg, soy and wheat, while allergies to peanuts, tree nuts, fish and shrimp usually are not outgrown. Exemplary milk associated allergens are casein, β-lactoglobulin and bovine serum albumin. Panels of the aforementioned allergens are readily available from sources such as Brendan BioScience, LLC (Boston, Mass.)

The information gleaned from using the devices and methods of the invention will allow the clinician to arrive at conclusions with respect to a patient's previous hypersensitivity reaction or their susceptibility to further reactions. According to the Gell/Combs classification, there are four types of hypersensitivity reactions which contain all accepted immune reactions causing human and animal sensitivity to antigens:

Type I, is antibody-mediated (IgE) and is commonly called immediate hypersensitivity because the allergic reaction occurs in less than two hours post allergen exposure. IgE circulates in blood as a free molecule or bound to mast cells and basophils in tissue with a half-life of about two days or two weeks, respectively. When cell-bound IgE binds the allergen, a cascade of events occurs that ultimately leads to (rapid) release of vasoactive mediators, i.e., histamine, that result in clinical symptoms related to allergy. Cell-bound IgE is detected by skin testing, whereas circulating (free) IgE is measured in serum or plasma.

Type II, is also antibody-mediated (IgG, IgM, IgA) and is commonly called delayed hypersensitivity (DTH) because the allergic reaction occurs up to several days post allergen exposure. Type II hypersensitivity occurs when antibody binds to either self-antigen or foreign antigen on cells, and leads to phagocytosis, killer cell activity or complement-mediated lysis. Since IgM is not produced after sensitization, IgG and IgA are the primary mediators of Type II DTH reactions. These antibodies, alone or in combination, bind to either self or foreign antigen on cells (opsonization) leading to phagocytosis, killer cell activity or complement-mediated lysis. IgG activates complement (C3b) leading to formation of the membrane-attack-complex and cell lysis, whereas IgA does not activate complement and is not involved in cell lysis. The type of sample used to measure these antibodies e.g., serum vs. saliva, is very important. For example, after submucosal allergen exposure both IgG and IgA are found in blood, whereas only mucosal IgA is produced in secretory secretions such as saliva. However, topical or intraepithelial exposure to allergens results in secretory IgA in the absence of appreciable IgA or IgG in blood. Based on this, one may want to test for IgA in saliva and gut mucosa, as well as, IgG and IgA in serum if there is a history of exposure to ingested allergens, for example. Recent data shows that IgG binds to mast cells with a half-life of about 3 months. Further, when an allergen binds to IgG on mast cells an “IgE-like” release of vasoactive mediators occurs.

Type III, is antibody-mediated (IgG) and is also a delayed hypersensitivity (DTH) because the allergic reaction occurs days to weeks post allergen exposure. Type III hypersensitivity develops when immune complexes (IC) are formed in large quantities, or cannot be cleared adequately by the reticuloendothelial system. Allergen exposure results in production of IgG, which in turn binds to the allergen forming immune complexes (IC) in blood. IC activate complement resulting in covalent binding of C3b to IgG forming IC-C3b. IC-C3b binds to CR1 receptors on red blood cells (RBC). The RBCc release the IC-C3b in the liver and spleen and the IC-C3b are degraded. If IC are not cleared by RBC, IC deposit at various sites throughout the body. Damage ensues when IC deposit at a site, activate complement and release C3a. C3a causes leucocytes and mast cells to release proteases and vasoactive amines that damage blood vessels or other tissue components.

Type IV, is an entirely cell-mediated form of delayed hypersensitivity (DTH) as the allergic reaction occurs days to weeks post allergen exposure. The most serious DTH is Granulomatous, which occurs when macrophages (MΦ) ingest, but cannot degrade, an allergen resulting in persistent MΦ stimulation. Stimulated MΦ elaborate cytokines that cause the MΦ itself or other cell types to form granulomas. T cells are then stimulated by cytokines, which mediate the range of inflammation responses. There are four types of cell-mediated DTH reactions depending on the type of allergen, route of contact or ability to degrade an allergen. Clinically, the most relevant DTHs are Contact (epidermis) and Granulomatous (mucosa). Contact DTH occurs when a small molecule binds to skin proteins and activates T-cells. The T-cells release cytokines that make skin cells form a typical eczematous rash. For example, latex (medical gloves), nickel (jewelry) or urushiol (Poison Ivy) are small molecules that induce contact DTH. Irrespective of the cell type forming the lesion, T-cells play a major role. Type IV-DTH is diagnosed by exposing the skin or mucosa to allergenic challenge followed by visual exam of redness, swelling and induration.

“Immune- or Immunocomplexes (ICs)” as used herein refer to the aggregations of antibodies with allergen. ICs trigger the activation of the complement cascade. The mammalian complement system is a critical host defense mechanism comprising more than 25 proteins and cellular receptors. Red blood cells intercept complement associated ICs (e.g., IC-C3b) in the bloodstream and safely transport it to the liver. If a patient is C3b positive, ICs are not being cleared, which may lead to clinical symptoms related to allergy. The inventor has discovered that the clinician can make diagnoses on the basis of an identification and/or quantification of circulating allergen-specific ICs that are associated with C3b. It is important to note that the C3b protein can be cleaved into C3d and C3c subunits. Therefore, the invention envisages the detection of IC-C3b either directly, e.g. by an antibody that binds C3b or more preferably, by an antibody that specifically binds the C3d portion of C3b.

Complement system is composed of more than 25 different proteins produced by different tissues and cells including hepatocytes, macrophages and gut epithelial cells. These proteins are activated by a variety of agents and their activation proceeds in a cascade fashion leading to pathogen lysis. Gell-Coombs Class II and III reactions are mediated through the Classical (C1), Alternative (C3) and Lytic (C5-C9) complement pathways.

Classical pathway normally requires a suitable antibody (Ab, usually IgG) bound to antigen (Ag), complement components 1, 4, 2 and 3 and Ca++ and Mg++ cations. Binding of C1qrs (a calcium-dependent complex), present in normal serum, to Ag-Ab complexes results in autocatalysis of C1r. The altered C1r cleaves C1s and this cleaved C1s becomes an enzyme (C4-C2 convertase) capable of cleaving both C4 and C2. Activated C1s enzymatically cleaves C4 into C4a and C4b. C4b binds to the Ag-bearing particle or cell membrane while C4a remains a biologically active peptide at the reaction site. C4b binds C2 which becomes susceptible to C1s and is cleaved into C2a and C2b. C2a remains complexed with C4b whereas C2b is released in the micro environment. C4b2a complex, is known as C3 convertase in which C2a is the enzymatic moiety. C3 convertase, in the presence of Mg++, cleaves C3 into C3a and C3b. C3b binds to the membrane to form C4b2a3b complex whereas C3a remains in the micro environment. C4b2a3b complex functions as C5 convertase which cleaves C5 into C5a and C5b. Generation of C5 convertase marks the end of the classical pathway.

The alternative pathway begins with the activation of C3 and requires Factors B and D and Mg⁺⁺ cation, all present in normal serum. A metastable C3b-like molecule (C3i) is generated by slow hydrolysis of the native C3. C3i binds factor B which is cleaved by Factor D to produce C3iBb. C3iBb complex cleaves native C3 into C3a and C3b. C3b binds factor B, which is again cleaved by Factor D to produce C3bBb (C3 convertase). This C3 convertase (or the one generated by classical pathway: C4b2a), if not inactivated, will continue to act on C3 and cause its total depletion.

C3b, in fluid phase, is very short lived unless it finds a suitable stabilizing membrane or molecule (C3 activator). In the absence of exogenous pathogen, it binds quickly to autologous red cells via the C3b receptor, CR1 at a site close to decay accelerating factor (DAF) which prevents the binding of Factor B. Binding to CR1 also makes C3b susceptible to Factor I which cleaves it into many fragments (iC3b, C3c, C3d, C3e, etc.). C4b, generated in the classical pathway, is also regulated by DAF, CR1 and Factor I. A defect in or deficiency of DAF can lead to cell lysis and anemia, as in its absence further activation of C will proceed and lead to the membrane attack pathway (see below) and cell lysis.

Another serum protein, factor H, can displace factor B and bind to C3b. Binding of factor H makes C3b more susceptible to factor I. C3 convertase generated by the classical pathway is regulated also in a similar manner by DAF, Cr1 and Factor I. The only difference is that C4b-binding protein (C4b-BP, not factor H) makes it susceptible to Factor I. A genetic deficiency of factor I (or factor H) leads to uncontrolled C3 activation and is a major cause of inherited C3 deficiency.

Certain bacteria or their products (peptidoglycan, polysaccharides, etc.), provide a protected (activator) surface for C3b. Thus, C3b bound to such a surface is relatively resistant to the action of factor I. Even membrane bound C3bBb dissociates fairly rapidly. Stabilized C3 convertase cleaves more C3 and produces C3bBbC3b complex (analogous to C4b2a3b of the classical pathway), the C5 convertase which cleaves C5 into C5a and C5b. C5b initiates the membrane attack pathway which leads to cell lysis. While these pathways of C3 activation are initiated by different mechanisms, they are analogous to each other and both can lead to membrane lysis.

The alternative pathway provides a means of non-specific resistance against infection without the participation of antibodies and hence provides a first line of defense against a number of infectious agents. Many gram negative and some gram positive bacteria, certain viruses, parasites, heterologous red cells, aggregated immunoglobulins (particularly, IgA) and some other proteins (e.g. proteases, clotting pathway products) can activate the alternative pathway.

The lytic (membrane attack) pathway involves the C5-9 components. C5 convertase generated by the classical or alternative pathway cleaves C5 into C5a and C5b. C5b binds C6 and subsequently C7 to yield a hydrophobic C5b67 complex which attaches quickly to the plasma membrane. Subsequently, C8 binds to this complex and causes the insertion of several C9 molecules to bind to this complex and lead to formation of a hole in the membrane, resulting in cell lysis. The lysis of target cell by C5b6789 complex is nonenzymatic and is believed to be due to a physical change in the plasma membrane. C5b67 can bind indiscriminately to any cell membrane leading to cell lysis. Such an indiscriminate damage to by-standing cells is prevented by protein S (vitronectin) which binds to C5b67 complex and blocks its indiscriminate binding to cells other than the primary target.

As stated above, C3b is usually promptly cleared from the serum if it is not associated with a stabilizing molecule. The inventor notes that the presence of serum ICs, including C3b, is indicative of Class II and III allergic reactions, and as such, IC-C3b is the common junction for all three complement pathways and enables measurement of the complement component of Class II and III, reactions which will be missed if one measures antibody alone. Accordingly, the invention measures bound C3b (IC-C3b) through its binding to allergens in foods, chemicals, and food additives. Therefore, the invention involves a quantitative, semi-quantitative and/or qualitative assaying of IgG, IgA, IgM and/or IgE antibodies and common complement pathway IC-C3b produced as a result of exposure to food antigens (allergens), in a biological fluid.

The assays of the invention are capable of qualitatively and/or quantitatively measuring IgG, IgA, IgM and/or IgE antibodies and immunocomplexed C3b (IC-C3b) produced as a result of exposure to food. More preferably, the assays of the invention are capable of qualitatively and/or quantitatively measuring IgG and IC-C3b produced as a result of exposure to food. The antigens of interest are those consumed as food products or additives, i.e., allergens, which may cause chronic sensitivity and acute and chronic disease in humans and animals.

The devices and methods of the invention can be carried out in various combinations. For example, they can be used to determine the total amount of allergen-specific immunoglobulin and IC-C3b present in a biological sample and correlate the amount with the severity of hypersensitivity, for example. Moreover, the devices and methods may be used to identify the presence and/or quantify each allergen specific IgA, IgM, IgG, or IgE subtype within a biological sample one at a time or in combination within the same assay.

Additionally, if the source of the hypersensitivity is not known, the inventive assays can quickly be adapted to screen a wide range of allergens. Alternatively, individual allergen-specific immunoglobulin subtypes may be analyzed to classify a patients hypersensitivity according to the Gell/Combs scheme. The identification and/or quantification of allergen-specific immunoglobulins (Ig) of certain antibody subtypes as well as the identification and/or quantification of allergen-specific immunocomplexes comprising C3b, preferably allows the clinician to determine whether the patient is experiencing and/or has experienced, and/or is susceptible to a Type I, II, III or IV hypersensitivity reaction. Such classifications will allow the clinician to better assemble an appropriate treatment regimen.

For example, if the methods and devices disclosed herein identify and/or quantify allergen-specific IgE and substantially no IgG, IgA, IgM and immunocomplexed C3b (IC-C3b) in a biological sample from a patient that is or has experienced an allergic reaction to food or food additive, the clinician can make a diagnosis that the patient has experienced and is susceptible to a Type I hypersensitivity reaction. Alternatively, if the methods and devices disclosed herein identify and/or quantify allergen-specific IgG, IgM or IgA and immunocomplexed C3b (IC-C3b) but substantially no IgE, in a biological sample from a patient that is or has experienced an allergic reaction to food or food additive, the clinician can make a diagnosis that the patient has experienced and is susceptible to a Type II hypersensitivity reaction. Moreover, if the methods and devices disclosed herein identify and/or quantify primarily allergen-specific IgG and allergen-specific immunocomplexed C3b (IC-C3b) and substantially no allergen-specific IgE, IgA, or IgM in a biological sample from a patient that is or has experienced an allergic reaction to food or food additive, the clinician can make a diagnosis that the patient has experienced and is susceptible to a Type III hypersensitivity reaction. Finally, if the methods and devices disclosed herein identify and/or quantify substantially no allergen-specific immunoglobulins or immunocomplexed C3b (IC-C3b) in a biological sample from a patient that is or has experienced an allergic reaction to food or food additive, the clinician can make a diagnosis that the patient has experienced and is susceptible to a Type IV hypersensitivity reaction.

The invention measures allergen-specific IgG, IgA, IgM and/or IgE antibodies and IC-C3b produced as a result of exposure to food antigens using immunoassay tests such as ELISA or dipsticks. The measurement of IC-C3b in addition to allergen specific antibody, particularly IgG, in the same test improves the detection of food antigens over that of measuring antibodies alone. The antigens of interest, i.e., allergens, are those consumed as food products, which may cause chronic sensitivity and acute and chronic disease in humans and animals. Importantly, the invention detects the immune reactions of antibody and complement which comprise Gell-Coombs Classes I, II and III whereas, measuring antibody levels alone is only capable of detecting Class I and II hypersensitivity. Class II and III reactions are mediated through the Classical (C1), Alternative (C3) and Lytic (C5-C9) complement pathways. The ability to detect IC-C3b enables measurement of the complement component of Class II and III reactions which will be missed if one measures antibody alone.

Preferably, the assaying of IgG, IgA, IgM and/or IgE antibodies and IC-C3b is carried out using an immunoassay. The immunoassay may be a competitive immunoassay or non-competitive sandwich-type assay. Additionally, the assay may be carried out in a wet or “dry chemistry” solid-state format.

One aspect of the invention utilizes Enzyme-Linked Immunosorbent Assay (ELISA) methodology. Generalized ELISA procedures are well known in the art and can readily be adapted to test for the combination of allergen-specific IgG, IgA, IgM and/or IgE antibodies and IC-C3b, described herein. Preferably, a biological sample, e.g., serum, suspected of containing allergen-specific IgG, IgA, IgM and/or IgE antibodies and IC-C3b is applied to the solid phase upon which an allergen is immobilized. Following a brief period of incubation, the solid phase is rinsed and a combination of labeled binding partners is added that specifically bind the allergen-specific IgG, IgA, IgM and/or IgE antibodies and C3b, thereby forming a sandwich. Preferably, the combination of labeled binding partners contains a mixture of labeled immunoglobulins, e.g. anti-IgG, anti-IgA, anti-IgM, anti-IgE, and anti-C3d, whereby each individual labeled immunoglobulin specifically binds either an IgG, IgA, IgM or IgE antibody or C3d, respectively. Following a second rinsing, the amount of label bound to the solid state is determined and is proportional to the amount of respective allergen-specific IgG, IgA, IgM and/or IgE antibodies and IC-C3b present in the biological sample that was bound by labeled binding partners. As such, the label intensity reflects the amount of tested allergen-specific Ig and IC-C3b present in the biological sample. It should also be kept in mind that the allergen-specific Ig detected at the solid phase will only reflect the allergen-specific Ig that is capable of being bound by whichever labeled binding partner is used. For example if labeled anti-IgG and anti-C3d are used, the amount of immobilized label will reflect the amount of allergen-specific IgG and IC-C3b in the biological sample.

In one specific embodiment, an ELISA is structured such that a combination of allergens derived from Apple, Corn, Oat, Soybean, Baker's Yeast, Cottonseed, Onion, Strawberry, Banana, Cow's Milk, Orange, Sunflower Seed, Beef, English Walnut, Peanut, Tea, Beet, Garlic, Pork, Tomato, Brewer's Yeast, Grapefruit, Red Pepper, Tuna, Broccoli, Green Olive, Rice, Turkey, Cocao, Hops, Rye, White Potato, Cocoanut, Lemon, Safflower Seed, White Seedless Grape, Coffee, Mushroom, Sesame, Whole Egg (Chicken), Cola Nut, Mustard, Sole, Whole Wheat, Almond, Cherry Green Pea, Pineapple, Apricot, Chicken, Honeydew Melon, Pinto Bean, Barley, Chili Pepper, Lamb, Pumpkin, Basil, Cinnamon, Lettuce, Salmon, Beet, Clam, Lima Bean, Scallops, Cabbage, Crab, Lobster, Shrimp, Cantaloupe, Cranberry, Millet, Squash Mix, Carrot, Cucumber, Oregano, Sweet Potato, Cashew Nut, Dill Seed, Peach, Tumeric, Cauliflower, Ginger, Pear, Vanilla, Celery, Green Bean, Pecan, and Watermelon are immobilized. A biological sample, suspected of containing allergen-specific IgG antibodies and IC-C3b is applied to the solid phase. Following a brief period of incubation, the solid phase is rinsed and anti-IgG and anti-C3d labeled binding partners are added that will specifically bind any IgG antibodies and C3d, respectively, thereby forming a sandwich. Following a second rinsing, the amount of label bound to the solid state is determined and is proportional to the amount of allergen-specific IgG antibodies and IC-C3b present in the biological sample.

If there is no allergen-specific IgG, IgA, IgM and/or IgE antibodies and IC-C3b present in the biological sample, then no label will be immobilized on the solid phase and will be washed away.

As used herein, “substantially no” refers to almost no detectable allergen-specific immunoglobulin or immunocomplex relative to an amount of strongly detectable allergen-specific immunoglobulin or immunocomplex. For example, the presence of a “background” level of label development would be considered by the skilled artisan to constitute substantially no allergen-specific IgG, IgA, IgM and/or IgE antibodies and IC-C3b present in the biological sample. Additionally, the amount of immobilized label associated with detectable immunoglobulin or immunocomplex will be about 10 times, preferably about 100 times or more preferably about 1000 times more intense than immobilized label associated with “substantially no” immunoglobulin or immunocomplex.

Another aspect of the invention relates to an immunoassay carried out on a solid support, e.g., a dipstick. Preferably, the solid support is made of a biblious material such as nitrocellulose, for example, through which a biological fluid can migrate by capillary action. The bibulous material can be a single structure such as a sheet cut into strips or it can be particulate material bound to a support or solid surface such as found, for example, in thin-layer chromatography. The support for the bibulous material, where a support is desired or necessary will normally be water insoluble, non-porous, and rigid and usually will be of the same length and width as the bibulous strip but may be larger or smaller. A wide variety of organic and inorganic materials, both natural and synthetic, and combinations thereof, may be employed provided only that the support does not interfere with the capillary action of the strip, or non-specifically bind assay components, or interfere with the signal producing system. Illustrative polymers include polyethylene, polypropylene, poly(4-methylbutene), polystyrene, polymethacrylate, poly(ethylene terephthalate), nylon, poly(vinyl butyrate), glass, ceramics, metals, and the like.

Preferably, the dipstick has three zones: a first mobilizable zone, a second trap zone and a third zone arranged so that the first mobilization zone and the third zone are spaced apart by the second trap zone. A basic immunoassay test strip systems are disclosed in U.S. Pat. Nos. 6,001,658; 4,540,659; 4,740,468; 5,451,504 and as well as U.S. Pat. No. 4,956,275; European Patent Application 0 267 066; European Patent Application 0 381 173; U.S. Pat. Nos. 4,959,307; 4,960,691; 4,968,604; 4,952,520; PCT 87/02774; U.S. Pat. Nos. 4,963,468; 4,981,786; European Patent Application 0 383 619; U.S. Pat. Nos. 4,313,734; 4,373,932; 4,956,302; 4,624,929; 3,884,641; 4,965,047; 4,770,853; 5,256,372; 4,857,453; 5,145,789; 4,980,298; 3,399,204; 3,420,205; 4,066,646; 5,120,643; 4,447,192; European Patent Application 0 349 295; European Patent Application 0 306 772; European Patent Application 0 299 428; PCT Application 93/03175; European Patent Application 0 291 194; European Patent Application 0 271 204; and European Patent Application 0 323 605, the test strip may be configured in any appropriate fashion, for any appropriate test, to include alternatives of any one or more of the above-described variants. A detailed discussion of these many variants for suitable test strips appears in the above listed documents, the entire contents of which are hereby fully incorporated by reference. Optionally, the solid support device may be inserted into a holder, such as disclosed in U.S. patent application Ser. No. 08/476,036 to MacKay et al., filed Jun. 7, 1995, whose contents are fully and totally incorporated herein by reference.

A preferred non-competitive test strip immunoassay embodiment provides for moving a biological sample suspected of containing an allergen-specific IgG, IgA, IgM and/or IgE antibodies and/or IC-C3b through a first mobilization zone, a second trap zone, and a third detection zone. At least two types of diffusible labeled receptors are provided on the first zone: a first type being specific for IgG, IgA, IgM and/or IgE antibodies and a second type being specific for C3b or more preferably the C3d portion of C3b. The second zone has at least one area having at least one immobilized allergen and the third zone provides for a control as it contains an immobilized second receptor specific for at least one of the diffusible labeled receptor types. When the dipstick is brought into contact with a biological sample such as serum, the liquid first flows through the first zone mobilizing the diffusible labeled receptors specific to IgG, IgA, IgM and/or IgE antibodies and C3b (or C3d portion of C3b).

Once mobilized, the diffusible labeled receptors will bind any IgG, IgA, IgM and/or IgE antibodies and C3b (or C3d portion of C3b) present in the biological sample to form a mobile labeled complex. The mobile labeled complex will in turn bind the at least one immobilized allergen located in the second zone to form an immobilized sandwich that can be visualized by the label. The remainder of unbound diffusible labeled receptors continues to migrate to the third control zone where an immobilized second receptor specifically binds and immobilizes some of the diffusible labeled receptors. Label development at the control zone demonstrates that fluid has properly migrated through the dipstick.

If there are substantially no allergen-specific IgG, IgA, IgM or IgE antibodies or IC-C3b present in the biological sample, there will be no immobilization of the diffusible labeled receptors and substantially no label at the second trap zone. Therefore, a negative result will result in label only accumulating at the control zone. Accordingly, it should be clear to one of skill in the art that the amount of label at the second zone is proportional to the amount of allergen-specific IgG, IgA, IgM and/or IgE antibodies and IC-C3b present in the sample. The allergen-specific Ig detected at the second zone will only reflect the allergen-specific Ig that is capable of being bound by which ever labeled receptor is used. For example if labeled anti-IgG and anti-C3d are used, the amount of immobilized label at the second zone will reflect the amount of allergen-specific IgG and IC-C3b in the biological sample. Alternatively, if the sample is a human sample and labeled anti-human Ig and anti-C3d are used as diffusible labeled receptors for example, the amount of immobilized label at the second zone will reflect the amount of all allergen-specific Ig as well as IC-C3b in the biological sample.

In the preferred embodiment, the first zone contains a mixture of labeled antibodies derived from goat that are specific for either anti-IgG, anti-IgA, anti-IgM and/or anti-IgE antibodies and anti-C3b antibodies. Most preferably, there are two species of diffusible labeled receptors: gold-conjugated goat anti-human IgG antibody and gold-conjugated goat anti-human C3d antibody. For this embodiment, the preferred immobilized second receptor specific for the diffusible labeled receptors at the third zone is a mouse generated anti-goat antibody.

Furthermore, the preferred immobilized allergen at the second trap zone is food derived allergen. Even more preferable is the subdivision of the second zone into discrete areas, such as stripes, each having a different allergen immobilized thereon. This enables the technician to discern among multiple allergen specific-IgG, IgA, IgM or IgE antibodies or IC-C3b in the biological sample. This will help the clinician quickly narrow down which allergen and, by extension, which food product or additive, is causing a patient's hypersensitivity response.

Preferably, the first diffusible labeled receptor is an antibody that is labeled with either enzymes, fluorophores, chromophores, radioisotopes, dyes, colloidal pigments or gold, latex particles, or chemiluminescent agents.

In all embodiments of the invention, the label may be directly visible, such as by the use of colloidal particles, e.g. gold and pigments, or latex microparticles. Alternatively, the label may be part of a signal producing system. The signal producing system may have one or more components, at least one component being the label conjugated to an receptor. The signal producing system includes all of the reagents required to produce a measurable signal. Other components of the developer include substrates, coenzymes, enhancers, second enzymes, activators, cofactors, inhibitors, scavengers, metal ions, specific binding substances required for binding of signal generating substances, and the like. The components of the signal producing system may be bound to the strip such as coenzymes, substances that react with enzymatic products, other enzymes and catalysts, and the like. The signal producing system provides a signal detectable by external means, normally by measurement of electromagnetic radiation, desirably by visual examination. For the most part, the signal producing system includes a chromophoric substrate and enzyme, where chromophoric substrates are enzymatically converted to dyes which absorb light in the ultraviolet or visible region, phosphors or fluorophores.

The skilled artisan will also appreciate that the invention may readily be adapted for use on non-human animals, particularly domesticated animals.

### EXAMPLE 1

An aliquot of blood is drawn in a standard blood withdrawing tube. The sample serum or saliva is isolated by centrifugation, diluted, and delivered to microtiter wells. The wells are coated with specific antigens of individual foods, food additives, and chemicals. Patient serum is added to the wells and incubated with anti-IgG, IgA, IgM, IgE and C3d. Binding occurs when the patient sera contains antibodies to the specific antibodies on the plate. Since C3b is central to all three compliment pathways, C3b binding indicates the activation of the complement cascade. A ligand, which binds to the anti-antibodies, develops a color only when the individual's serum antibodies and complement attach to the plated antigens.

The test is accomplished by coating individual wells of a micro titer plate with specific and different food, chemical, and additive antigens. The plate is then blocked with bovine serum albumin (BSA). Serum is added and incubated. An anti-IgG, IgA, IgM, IgE and C3d is applied and washed. After incubation, this sample is treated with color developer. Color develops proportionally to the amount of antibody/antigen binding that occurs. In addition to anti-IgG, IgA, IgM and IgE, anti-C3d is incorporated to measure immune complex formation. The combination of multiple antibodies and complement components C, C3, C3b and C3d and other complement components that are commercially available can be used in this test.

Adequate negative and positive controls will be determined on each plate or strip to confirm a meaningful, accurate result.

### EXAMPLE 2

A second example uses a nitrocellulose strip as a dipstick or lateral flow to give a qualitative response to food, additive and chemical sensitivity.

A nitrocellulose membrane is striped with a combination of specific antigens With the appropriate negative control, several foods and other antigens can be tested on each membrane strip. Patient sera is collected and mixed with buffered diluent. The diluted sample is added to a tube or a device which contains a colloidal label linked to anti-IgE, -IgA, -IgM, and -C3d. The strip is dipped into sera containing the colloidal labeled anti-IgE, -IgA, -IgM, and -C3d or sera is dropped onto the lateral flow device containing mobilizable colloidal labeled anti-IgE, -IgA, -IgM, and -C3d. When patient sera containing antibody and complement binds to the striped antigen, a red line develops. This is a low cost qualitative indication of sensitization to foods, chemicals and food additives.

Allergenic protein extracts from foods, chemicals and food additives will be coated (striped) onto the surface of a nitrocellulose membrane and used to capture human antibodies: IgM, IgG, IgA, IgE and C3d from human or animal serum or plasma. It is envisioned that the first prototype test will employ two proteins from the above list. A procedural control, such as anti-goat IgG, will be coated (striped) in a separate line on the nitrocellulose membrane. Thus a total of three lines will be present on the nitrocellulose membrane. This membrane will be housed in a plastic device that has a secondary reagent, such as Colloidal Gold-(Goat-anti-Human IgM/IgG, H&L), dried onto the surface of a glass pad. The sample will be added through a window in the housing, contact the CG-(anti-Human IgM/IgG, H&L) and form the complex CG-(anti-Human IgM/IgG, H&L)-Human IgM/IgG. This complex will flow along the strip and contact the allergen proteins (the first two lines) and, subsequently, contact the anti-Goat IgG (control). If one or both of the first two lines form and the control line forms, the sample is positive. If only the control line forms, the sample is negative. The total test time will be approximately 5 minutes from sample to result. The sensitivity and specificity of the Lateral Flow Test can be determined by comparison to the ELISA data.

In this disclosure there are described only the preferred embodiments of the invention and but a few examples of its versatility. It is to be understood that the invention is capable of use in various other combinations and environments and is capable of changes or modifications within the scope of the inventive concept as expressed herein. Thus, for example, those skilled in the art will recognize, or be able to ascertain, using no more than routine experimentation, numerous equivalents to the specific substances and procedures described herein. Such equivalents are considered to be within the scope of this invention.

1. A kit for determining the presence of allergen-specific immunoglobulins and immuncomplex C3b (IC-C3b) in a biological sample comprising:
(a) a solid support comprising an immobilized allergen that is to be exposed to a biological sample thereby binding and immobilizing allergen-specific immunoglobulins and immuncomplex C3b; and
(b) at least two labeled binding partners:
(i) a first labeled binding partner that specifically binds the immobilized allergen-specific immunoglobulins; and
(ii) a second labeled binding partner that specifically binds the immobilized immunocomplex C3b.

2. The kit of claim 1 wherein said biological sample is serum or saliva.

3. The kit of claim 1 wherein said solid support is a microtiter dish well.

4. The kit of claim 1 wherein said first labeled binding partner is selected from the group consisting of labeled anti-human, anti-IgG, anti-IgA, and anti-IgM antibodies and said second labeled binding partner is an anti-C3d antibody.

5. The kit of claim 1 wherein said first labeled binding partner is anti-IgG antibody and said second labeled binding partner is an anti-C3d antibody.

6. The kit of claim 1 wherein said label is part of a signal producing system.

7. The kit of claim 1 wherein the amount of label immobilized on the solid support can be read quantitatively.

8. The kit of claim 1 wherein the allergen is derived from the group consisting of milk, corn, shrimp, lobster, crab, peanuts, walnuts, fish, eggs, soy and wheat.

9. The kit of claim 1 wherein the allergen is selected from the group consisting of monosodium glutamate (MSG), gluten, casein, β-lactoglobulin and bovine serum albumin.

10. A method of determining the presence of allergen-specific immunoglobulins and immuncomplex C3b (IC-C3b) in a biological sample comprising:
(a) exposing a solid support comprising an immobilized allergen to a biological sample;
(b) washing unbound molecules from the biological samples from the solid support;
(c) exposing said solid support to at least two labeled binding partners:
(i) a first labeled binding partner that specifically binds the immobilized allergen-specific immunoglobulins; and
(ii) a second labeled binding partner that specifically binds the immobilized immuncomplex C3b;
(d) washing unbound labeled binding partners from the solid support;
(e) detecting the presence of label bound to the solid support; and
correlating it with the presence of allergen specific IC-C3b and allergen specific immunoglobulins in the biological sample.

11. The method of claim 10 wherein said biological sample is serum or saliva.

12. The method of claim 10 wherein said solid support is a microtiter dish well.

13. The method of claim 10 wherein said first labeled binding partner is selected form the group consisting of labeled anti-human, anti-IgG, anti-IgA, and anti-IgM antibodies and said second labeled binding partner is an anti-C3d antibody.

14. The method of claim 10 wherein said first labeled binding partner is anti-IgG antibody and said second labeled binding partner is an anti-C3d antibody.

15. The method of claim 10 wherein said label is part of a signal producing system.

16. The method of claim 10 wherein the amount of label immobilized on the solid support is be read quantitatively.

17. The method of claim 10 wherein the allergen is derived from the group consisting of milk, corn, shrimp, lobster, crab, peanuts, walnuts, fish, eggs, soy and wheat.

18. The method of claim 10 wherein the allergen is selected from the group consisting of monosodium glutamate (MSG), gluten, casein, β-lactoglobulin and bovine serum albumin.

19. The method of claim 10 wherein:
A. a presence of allergen-specific IgE and a substantial lack of allergen-specific IgG, IgA, IgM and IC-C3b in the biological sample correlates with Type I hypersensitivity reactions;
B. a presence of allergen-specific IgG, IgA, and IgM and IC-C3b and a substantial lack of allergen-specific IgE in the biological sample correlates with Type II hypersensitivity reactions;
C. a presence of allergen-specific IgG and IC-C3b and a substantial lack of allergen-specific IgE, IgA, IgM in the biological sample correlates with Type I hypersensitivity reactions; and
D. a substantial lack of allergen-specific IgG, IgA, IgM and IC-C3b in the biological sample correlates with Type I hypersensitivity reactions.
